(19) United States
(12) Patent Application Publication
(10) Pub. No.: US 2013/0190606 A1
(43) Pub. Date: Jul. 25, 2013

(54) COMBINED QUANTITATIVE MRI AND QUANTITATIVE MRS FOR DIAGNOSIS OF ALZHEIMERS DISEASE AND HIPPOCAMPAL SCLEROSIS

(71) Applicant: Huntington Medical Research Institutes, Pasadena, CA (US)

(72) Inventor: Brian D. Ross, Altadena, CA (US)

(73) Assignee: Huntington Medical Research Institutes, Pasadena, CA (US)

(21) Appl. No.: 13/750,901

(22) Filed: Jan. 25, 2013

Related U.S. Application Data
(60) Provisional application No. 61/590,646, filed on Jan. 25, 2012.

(51) Int. Cl. A61B 5/055; A61B 5/00

(52) U.S. Cl. CPC A61B 5/055; A61B 5/4848
USPC 600/411; 600/410

(57) ABSTRACT

Using both quantitative MRI and quantitative MRS, the inventor demonstrates herein that as age and dementia progress, hippocampal volume (HV), amygdala volume (AV), and NAA/mI ratio all decrease compared to normal controls. The inventor's data also demonstrate herein that NAA/mI vs. total amygdala volume showed a very strong linear correlation, while NAA/mI vs. mean total hippocampal volume had a lesser but still significant correlation. Further, after analyzing the data derived from both quantitative MRI and quantitative MRS methods, the inventor made the surprising discovery that the two tests, when combined, provide significantly improved biomarkers over either alone, and are more effective at early MCI and/or AD diagnosis, as well as HS diagnosis.

### CROSS-REFERENCE TO RELATED APPLICATIONS

This application claims the benefit of U.S. Provisional Patent Application No. 61/590,646, filed on Jan. 25, 2012, which is incorporated herein by reference in its entirety as though fully set forth.

### FIELD OF THE INVENTION

The present invention relates generally to methods for detection of Alzheimer's disease (AD), mild cognitive impairment (MCI), and hippocampal sclerosis (HS).

### BACKGROUND

All publications herein are incorporated by reference to the same extent as if each individual publication or patent application were specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Although reduction in brain volume can occur without significant cognitive impairment, recent Alzheimer's Disease Neuroimaging Initiative (ADNI) studies validate reductions in hippocampal and amygdala volume as biomarkers for mild cognitive impairment (MCI) and Alzheimer's disease (AD). Meanwhile, preservation of neurological function and therefore ‘normal’ neurochemistry in the compressed brain of a successful mathematician is best explained by rebalancing of cerebral osmolytes after shunt-treatment of hydrocephalus.

Considering the prevalence of Alzheimer's disease, and the limitations of traditional diagnostic methods, there is a need in the art for improved early detection methods, incorporating the latest advancements in MRI and MRS techniques to accurately measure anatomical changes in the brain, as well as chemical changes.

As demonstrated herein, the anatomical and chemical changes in the brain measurable by MRI and MRS are not only useful for the diagnosis of Alzheimer's, but also for differentiating between Alzheimer's and other conditions which are commonly misdiagnosed as Alzheimer's, such as mild cognitive impairment (MCI) and hippocampal sclerosis (HS).

### SUMMARY OF THE INVENTION

In some embodiments, the invention teaches a method for diagnosing an individual with Alzheimer's disease or mild cognitive impairment, including: determining a hippocampal volume (HV) and/or an amygdala volume (AV) in the brain of the individual; determining the ratio of N-acetylasparate (NAA) to myo-inositol (mI) in the brain of the individual; and diagnosing the individual with Alzheimer's disease or mild cognitive impairment if the HV and/or AV and/or NAA to mI ratio is below that of a healthy individual without Alzheimer's disease or mild cognitive impairment, or diagnosing the individual as not having Alzheimer's disease or mild cognitive impairment if the HV and/or AV and/or NAA to mI ratio is not below that of a healthy individual without Alzheimer's disease or mild cognitive impairment. In some embodiments, the HV and AV are determined using quantitative magnetic resonance imaging. In some embodiments, the NAA to mI ratio is determined using quantitative magnetic resonance spectroscopy.

In some embodiments, the invention teaches a method for diagnosing the progression of Alzheimer's disease or mild cognitive impairment in an individual, including: determining a hippocampal volume (HV) and/or an amygdala volume (AV) in the brain of the individual; determining the ratio of N-acetylasparate (NAA) to myo-inositol (mI) in the brain of the individual; and diagnosing the individual as having progressing Alzheimer's disease if the HV and/or AV and/or NAA to mI ratio are lower than previously determined in the same individual, and diagnosing the individual as not having progressing Alzheimer's disease if the HV and/or AV and/or NAA to mI ratio are not lower than previously determined in the same individual. In some embodiments, the HV and AV are determined using quantitative magnetic resonance imaging. In some embodiments, the NAA to mI ratio is determined using quantitative magnetic resonance spectroscopy.

In some embodiments, the invention teaches a method for determining the efficacy of a drug for treating or preventing Alzheimer's disease (AD) or mild cognitive impairment (MCI) in an individual, including: determining a hippocampal volume (HV) and/or amygdala volume (AV) in the brain of the individual; determining the ratio of N-acetylasparate (NAA) to myo-inositol (mI) in the brain of the individual; providing a dose of a drug for treating or preventing Alzheimer's disease (AD) or mild cognitive impairment (MCI); administering the dose of the drug for treating or preventing AD or MCI to the individual; determining the hippocampal volume (HV) and/or amygdala volume (AV) after administering the drug for treating or preventing AD or MCI to the individual; determining the ratio of NAA to mI after administering the drug for treating or preventing AD or MCI to the individual; comparing the HV and/or AV and/or NAA to mI ratio determined prior to administering the drug with the HV and/or AV and/or NAA to mI ratio determined after administering the drug; and determining the drug has efficacy for treating or preventing AD or MCI if the HV and/or AV and/or NAA to mI ratio has not decreased significantly after administering the drug, and determining the drug does not have efficacy for treating or preventing AD or MCI if the HV and/or AV and/or NAA to mI ratio has decreased significantly after administering the drug. In some embodiments, the drug is selected from the group consisting of a cholinesterase inhibitor and an N-methyl-D-aspartate (NMDA) receptor antagonist. In some embodiments, the drug is selected from the group consisting of donepezil, rivastigmine, galantamine, tacrine, and memantine. In some embodiments, the HV and AV are determined using quantitative magnetic resonance imaging. In some embodiments, the NAA to mI ratio is determined using quantitative magnetic resonance spectroscopy.

In some embodiments, the invention teaches a method for determining whether an individual should be included in or excluded from a clinical trial based upon diagnosis or exclusion of Alzheimer's disease or mild cognitive impairment at an early stage, including: determining a hippocampal volume (HV) and/or an amygdala volume (AV) in the brain of the individual; determining the ratio of N-acetylasparate (NAA) to myo-inositol (mI) in the brain of the individual; diagnosing the individual with Alzheimer's disease or mild cognitive impairment if the HV and/or AV and/or NAA to mI ratio is below that of a healthy individual without Alzheimer's disease or mild cognitive impairment at an early stage, or diagnosing the individual as not having Alzheimer's disease or mild cognitive impairment if the HV and/or AV and/or NAA to mI ratio is not below that of a healthy individual without Alzheimer's disease or mild cognitive impairment at an early stage; and determining the individual should be included in the clinical trial if the individual is diagnosed with Alzheimer's disease or mild cognitive impairment at an early stage, and determining the individual should be excluded from the clinical trial if the individual is not diagnosed with Alzheimer's disease or mild cognitive impairment at an early stage. In some embodiments, the HV and AV are determined using quantitative magnetic resonance imaging. In some embodiments, the NAA to mI ratio is determined using quantitative magnetic resonance spectroscopy.

In some embodiments, the invention teaches a method for diagnosing an individual with hippocampal sclerosis (HS), including: determining a hippocampal volume (HV) in the brain of the individual; determining the ratio of N-acetylasparate (NAA) to myo-inositol (mI) in the brain of the individual; and diagnosing the individual with HS if the HV is below that of a healthy individual without HS, and the NAA to mI ratio is not below that of a healthy individual without HS. In some embodiments, the HV is determined using quantitative magnetic resonance imaging. In some embodiments, the NAA to mI ratio is determined using quantitative magnetic resonance spectroscopy.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, spectra from normal (age-matched) control (A) and AD subject (B) with NAA/Cr (dark shading) and mI/Cr (light shading) peaks labelled. Acquired with a 1.5T GE scanner from the posterior cingulate gyrus grey matter region of the brain (inset image). (C) A reconstruction of an MCI subject's hippocampus and amygdala.

FIG. 2 demonstrates, in accordance with an embodiment of the invention (A) NAA/mI and mean normalized hippocampus volume have high positive correlation, and (B) NAA/mI ratio and mean normalized amygdala volume have strong linear correlation.

FIG. 3 demonstrates, in accordance with an embodiment of the invention, changes in brain volume and brain chemistry are not necessarily dependent upon one other.

FIG. 4 demonstrates, in accordance with an embodiment of the invention, all spectra acquired from the posterior cingulate gyrus grey matter region. (A) Acquired pre-intake. (B) Acquired post-intake. (C) Acquired in a patient who consumed MSM.

FIG. 5 demonstrates, in accordance with an embodiment of the invention, data acquired on 3 different scanners: GE 1.5T (HMRI) and 3T (ISP) and Siemens 3T (U Kentucky). NAA/mI values are identical (GE 1.5T vs. 3T P>0.15; GE 3T vs Siemens 3T P>0.08). However, NAA/Cr is different between 1.5T and 3T (P<0.02) as well as mI/Cr for all scanners (GE 1.5T vs. 3T P<0.01; GE 3T vs Siemens 3T P<0.01).

FIG. 6 demonstrates, in accordance with an embodiment of the invention, a longitudinal chart over a period of 13 months demonstrating the positive change of NAA/Cr (+14% in GM; +18% in WM) 1 month after consumption of AB34.

FIG. 7 demonstrates, in accordance with an embodiment of the invention, neurochemical changes are defined earlier than changes in brain volume.

FIG. 8 demonstrates, in accordance with an embodiment of the invention, quantitative MRS and MRI can be used to distinguish between HS and AD, by evaluating mI/Cr and NAA/Cr ratios (“Chart 1”), HV (“Chart 2”), and HV vs. NAA/mI (“Chart 3”).

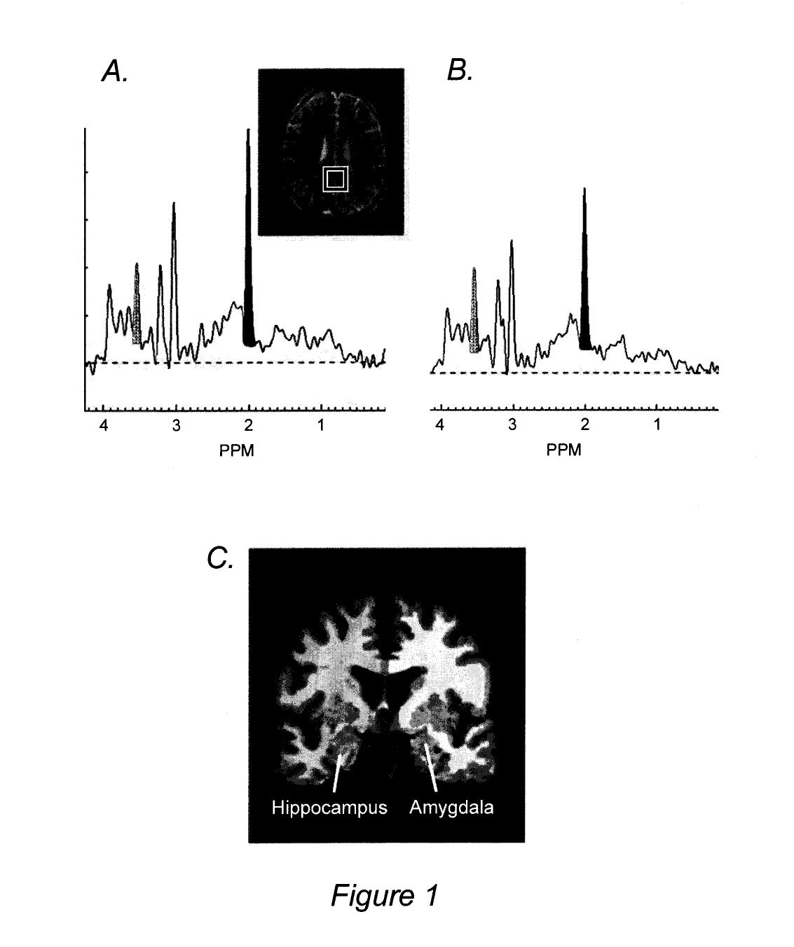

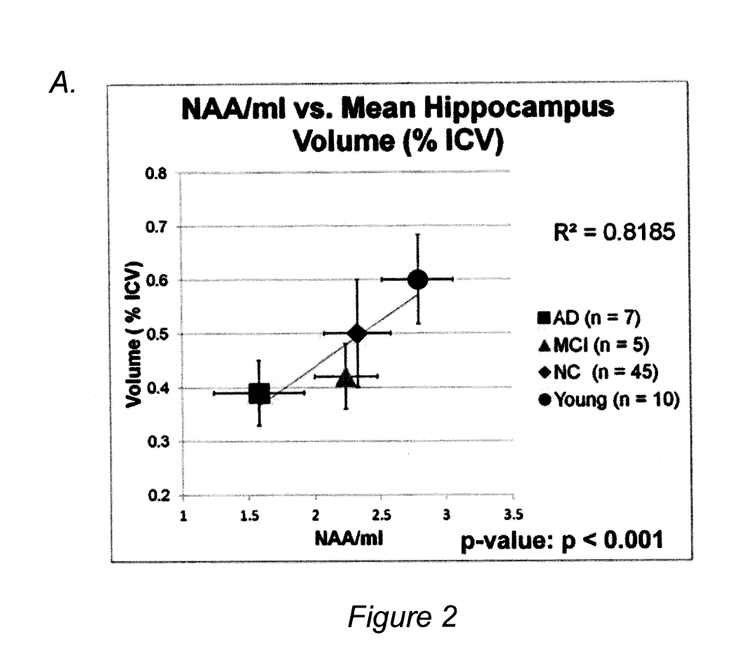

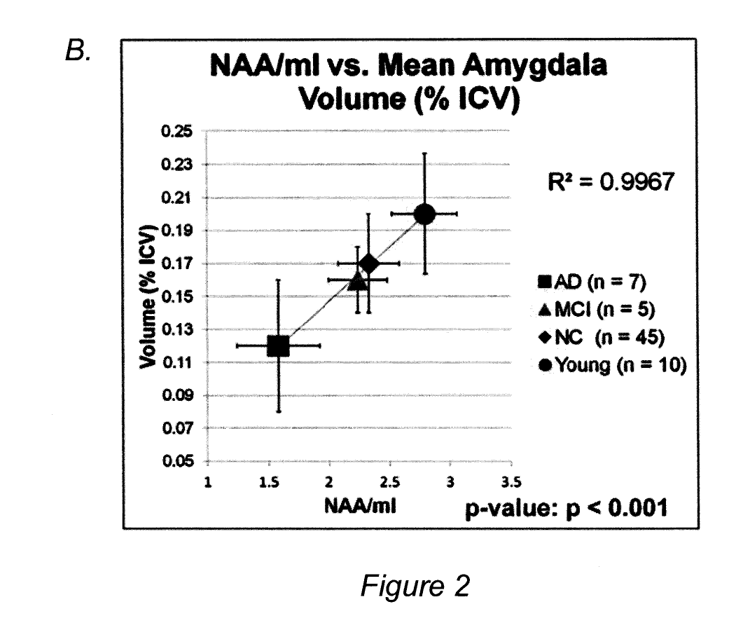

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons; and March, Advanced Organic Chemistry Reactions, Mechanisms and Structure 5^{th} ed., provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, certain terms are defined below.

As used herein, the acronym “AD” means Alzheimer's disease.

As used herein, the acronym “MCI” means mild cognitive impairment.

As used herein, the acronym “HS” means hippocampal sclerosis.

As used herein, the acronym “SDF” means stromal cell-derived factor.

As used herein, the abbreviation “NAA” means N-acetylaspartate.

As used herein, the abbreviation “mI” means myo-inositol.

As used herein, the acronym “HV” means hippocampal volume.

As used herein, the acronym “AV” means amygdala volume.

As used herein, the acronym “NC” means normal control.

By way of background, Alzheimer's disease was first described in 1907 by Alois Alzheimer. From its original status as a rare disease, Alzheimer's has become one of the most common diseases in the aging population, ranking as the fourth most common cause of death. Alzheimer's disease is a progressive neurodegenerative disorder characterized by the gradual onset of dementia. The pathologic hallmarks of the disease are beta-amyloid (Aβ) plaques, neurofibrillary tangles (NFTs), and reactive gliosis.

Current diagnosis of Alzheimer's disease is made by clinical, neuropyschological, and neuroimaging assessments. Routine structural neuroimaging evaluation is based on nonspecific features, such as atrophy, which, when evaluated using non-quantitative reading of brain images, can only be recognized as a late feature in the progression of the disease. Therefore, developing new approaches for earlier and more specific recognition of Alzheimer's disease is of crucial importance.

While historically there was believed to be a strong correlation between brain structure and function, particularly with regard to atrophy, magnetic resonance imaging (MRI) technology has only relatively recently evolved sufficiently to reliably quantify these changes in a manner that meaningfully contributes to early Alzheimer's disease diagnosis. Meanwhile, quantitative magnetic resonance spectroscopy methods, such as those described in U.S. Pat. No. 5,617,861, which is incorporated herein by reference in its entirety as though fully set forth, have also proven to be effective in early diagnosis when used to determine the concentration of N-acetylaspartate (NAA) and myo-inositol (mI), abnormal values of which have been strongly correlated with Alzheimer's disease.

Not surprisingly, drug development for treatment of AD and MCI has been both expensive and largely unsuccessful, using currently recognized clinical endpoints (mini-mental state exam, etc). While drug discovery needs additional thought, FDA, NIA and Pharma now recommend the use of objective disease end points (“biomarkers”) in place of clinical diagnosis as a means of reducing group size and therefore cost of trials. In fact, the Alzheimer's Disease Neuroimaging Initiative (ADNI) harnesses the above-mentioned reduction in brain volume (approximately 3.0-3.5% per year) during the evolution of AD, which is now recommended along with SDF biomarkers, beta amyloid, and Tau protein. Importantly, the use of MRS to detect NAA/mI is also slowly gaining acceptance, as this ratio falls by about 3.5% per year in those with Alzheimer's disease.

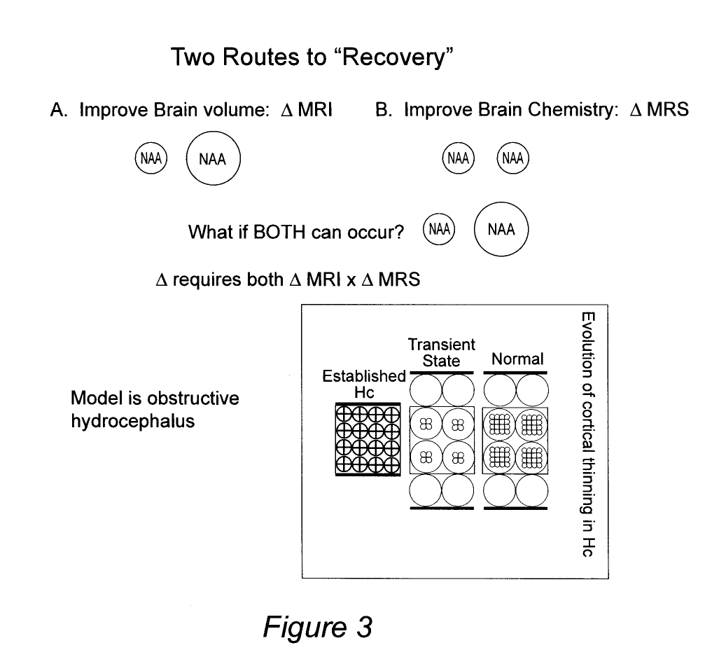

Although both quantitative MRI and quantitative MRS methods of early Alzheimer's disease detection are separately effective, their combined diagnostic value has not been previously investigated. This is probably the result of a number of considerations, including: (1) the above-mentioned MRS-based detection method is less well known/accepted than the more widely known/accepted MRI-based method, (2) a standard operating procedure for using quantitative MRS wasn't previously well-defined, and (3) those who do recognize the diagnostic value of quantitative MRS may consider it to be largely overlapping with what is currently achievable by quantitative MRI, based upon the incorrect assumption that reductions in brain volume and brain chemistry are necessarily dependent upon one another (see FIG. 3).

Using both quantitative MRI and quantitative MRS, the inventor demonstrates herein that as age and dementia progress, hippocampal volume (HV), amygdala volume (AV), and NAA/mI ratio all decrease compared to normal controls (Table I). The inventor's data also demonstrate herein that NAA/mI vs. total amygdala volume showed a very strong linear correlation, while NAA/mI vs. mean total hippocampal volume had a lesser but still significant correlation.

Further, after analyzing the data derived from both quantitative MRI and quantitative MRS methods, the inventor made the surprising discovery that the two tests, when combined, provide significantly improved biomarkers over either alone, and are consequently more effective at early MCI and/or AD diagnosis.

The inventor also discovered that the combined quantitative MRI and quantitative MRS methods described herein are not only useful in differentiating between AD and normal, and AD and MCI, but also AD and HS. This is an important discovery, because HS is a relatively common neuropathological finding (˜10% of individuals over the age of 85 years) characterized by cell loss and gliosis in the hippocampus that is not explained by AD. Although much remains to be learned about the disease, it is known that HS pathology can be associated with different underlying causes, including ageing.

Considering the common symptoms of AD and HS, it is not surprising that differentiating between the two can be challenging. While recent studies have shown that a neuropyschological profile that evaluates verbal fluency and word list recall can be helpful in differentiating between individuals with AD and HS, the objective biomarker-based test discovered by the inventor represents a significant advancement over traditional methods, allowing for convenient and accurate diagnosis of HS without relying on subjective criteria (see Nelson et al. Brain. 2011 May; 134 (Pt 5):1506-18, which is incorporated by reference herein in its entirety as though fully set forth). In summary, the inventive methods allow for the diagnosis of AD, HS, and MCI.

Therefore, in some embodiments, the invention teaches a method for diagnosing AD or MCI in an individual, including: determining a hippocampal volume (HV) and/or an amygdala volume (AV) in the brain of the individual; determining the ratio of N-acetylaspartate (NAA) to myo-inositol (mI) in the brain of the individual; and diagnosing the individual with AD or MCI if the HV and/or AV and NAA/mI ratio is below that of a healthy individual without AD or MCI, and not diagnosing the individual with AD or MCI if the HV and/or AV and NAA/mI ratio is not below that of a healthy individual without AD or MCI. In certain embodiments, HV and AV are determined using quantitative magnetic resonance imaging. In certain embodiments, the NAA/mI ratio is determined using quantitative magnetic resonance spectroscopy. In some embodiments the diagnostic method described above is used in conjunction with one or more additional methods of diagnosis, which may include but are in no way limited to medical history evaluation and mental status testing. In some embodiments, the individual is a male. In some embodiments, the individual is a female.

In certain embodiments, the present invention teaches treating an individual with one or more drugs, after that individual has been diagnosed with AD or MCI using the above-identified method. In some embodiments, one or more of the drugs used to treat the individual can include a cholinesterase inhibitor. In some embodiments, one or more of the drugs used to treat the individual can include an N-methyl-D-aspartate (NMDA) receptor antagonist. In certain embodiments, one or more of the drugs used to treat the individual can include donepezil, rivastigmine, galantamine, tacrine, memantine, or combinations thereof. In some embodiments, the patient is treated with a combination of an NMDA receptor antagonist and a cholinesterase inhibitor. In some embodiments, the patient is treated by administering vitamin E alone or in combination with one or more of the aforementioned drugs. In some embodiments, the treatment protocol is established according to the stage of AD or MCI determined by the inventive method described herein.

In certain embodiments, the invention teaches a method for diagnosing the progression of AD or MCI in an individual, including: determining a hippocampal volume (HV) and/or an amygdala volume (AV) in the brain of the individual; determining the ratio of N-acetylasparate (NAA) to myo-inositol (mI) in the brain of the individual; and diagnosing the individual as having progressing AD or MCI if the HV and/or AV and/or NAA to MI ratio are lower than previously determined in the same individual, and diagnosing the individual as not having progressing AD or MCI if the HV and/or AV and/or NAA to mI ratio are not lower than previously determined in the same individual. In some embodiments, the HV and AV are determined using quantitative magnetic resonance imaging. In some embodiments, the NAA/mI ratio is determined using quantitative magnetic resonance spectroscopy. In some embodiments, the method of diagnosing progression is used in conjunction with one or more additional methods of diagnosis described herein. In some embodiments, the patient is treated with one or more of the drugs (or classes of drugs) described above, selected according to the rate of disease progression and/or the stage of the disease, as determined by the inventive method.

In certain embodiments, the invention teaches a method for determining the efficacy of a drug for treating or preventing AD or mild cognitive impairment MCI in an individual, including: determining HV and/or AV in the brain of the individual; determining the ratio of NAA to mI in the brain of the individual; providing a dose of a drug for treating or preventing AD or MCI; administering the dose of the drug for treating or preventing AD or MCI to the individual; determining the HV and/or AV after administering the drug for treating or preventing AD or MCI to the individual; determining the ratio of NAA to mI after administering the drug for treating or preventing AD or MCI to the individual; comparing the HV and/or AV and/or NAA to mI ratio determined prior to administering the drug with the HV and/or AV and/or NAA to mI ratio determined after administering the drug; and determining the drug has efficacy for treating or preventing AD or MCI if the HV and/or AV and/or NAA to mI ratio has not decreased significantly after administering the drug, and determining the drug does not have efficacy for treating or preventing AD or MCI if the HV and/or AV and/or NAA to mI ratio has decreased significantly after administering the drug. In some embodiments, the efficacy of the drug is evaluated according to the inventive method after a period of time ranging from 1 day to 10 years after the treatment commences. In some embodiments, the efficacy of the drug is evaluated according to the inventive method after a period of time ranging from 7 days to 5 years after the treatment commences. In some embodiments, the efficacy of the drug is evaluated according to the inventive method after a period of time ranging from 30 days to 3 years after the treatment commences. In some embodiments, the efficacy of the drug is evaluated according to the inventive method after a period of time ranging from 60 days to 1 year after the treatment commences.

In some embodiments, the inventive method is used to evaluate the efficacy of a combination of drugs for treating or preventing AD or MCI. In some embodiments, one or more of the drugs are selected from any of the drugs or classes of drugs described herein. In some embodiments, one or more of the drugs is an experimental drug.

In certain embodiments, the invention teaches a method for determining whether an individual should be included in or excluded from a clinical trial, based upon diagnosis or exclusion of AD or MCI at an early stage, including: determining a HV and/or an AV in the brain of the individual; determining the ratio of NAA to mI in the brain of the individual; diagnosing the individual with AD or MCI if the HV and/or AV and/or NAA to mI ratio is below that of a healthy individual without AD or MCI at an early stage, or diagnosing the individual as not having AD or MCI if the HV and/or AV and/or NAA to mI ratio is not below that of a healthy individual without AD or MCI at an early stage; and determining the individual should be included in the clinical trial if the individual is diagnosed with AD or MCI at an early stage, and determining the individual should be excluded from the clinical trial if the individual is not diagnosed with AD or MCI at an early stage.

In various embodiments, the invention teaches a method for diagnosing an individual with HS, including: determining a HV in the brain of the individual; determining the ratio of NAA to mI in the brain of the individual; and diagnosing the individual with HS if the HV is below that of a healthy individual without HS, and the NAA to mI ratio is not below that of a healthy individual without HS. In some embodiments, the HV is determined using quantitative magnetic resonance imaging. In some embodiments, the NAA to mI ratio is determined using quantitative magnetic resonance spectroscopy.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed, while still obtaining a like or similar result, without departing from the spirit and scope of the invention.

### Example 1

### Experiments I

### Patients and Methods

Sixty-seven subjects (AD n=7 (age 72±10), MCI n=5 (age 79±4), Normal (Elderly) Control (age 76±6) n=45, Young Control (age 20±3) n=10) were scanned using a GE 1.5T scanner to obtain brain scans and ¹H MRS data. Each subject was categorized into one of the four groups based on his/her results from clinical work-up, mini-mental state examination (MMSE) scores and MRS results. Short TE MRS data was acquired using standard proton brain exam (PROBE-P) sequence employing the 8 cm³ voxel positioned in the grey matter region of the posterior cingulate gyrus for technical and diagnostic reasons (See Kantarci et al. Radiology (2008) 248(1): 210-220, which is incorporated by reference herein in its entirety as though fully set forth). Analysis of the neuronal marker N-acetylaspartate (NAA), which has been shown to decrease upon progression towards Alzheimer's, and glial marker myo-inositol (mI), which increases with advancing dementia, were performed (see Shonk T K, Moats R A, Ross B D. Radiology (1995) 195:65-72, which is incorporated herein by reference in its entirety as though fully set forth). Subcortical volumes were extracted using the automated MRI-reconstruction program FreeSurfer. Each reconstruction was made following FreeSurfer's recommended reconstruction workflow with manual adjustments performed as needed.

### Example 2

### Data Analysis

NAA/Cr and mI/Cr (PROBE-Q, GE) were expressed as NAA/mI. Hippocampus and amygdala volumes were normalized to intracranial volume in each subject.

### Example 3

### Statistics

Paired student t-tests were utilized to compare volumes and biomarker values between each subject. R² values were converted to obtain corresponding p-values.

### Example 4

### Results

As age and dementia progress, hippocampal volume (HV), amygdala volume (AV), and NAA/mI ratio all decrease compared to normal controls (NC) (Table 1). NAA/mI vs. mean total amygdala volume showed a very strong linear correlation, while NAA/mI vs. mean total hippocampal volume had a lesser but still significant correlation.

TABLE I

| | HV ± SD | | AV ± SD | | |
|---|---|---|---|---|---|
| | (cm³) | % HV (/ICV) | (cm³) | % AV (/ICV) | NAA/mI ± SD |
| AD (n = 7) | 5.71 ± 1.15 | 0.39 ± .06 | 1.86 ± 0.65 | 0.12 ± .04 | 1.58 ± 0.34 |
| MCI (n = 5) | 6.28 ± 0.84 | 0.42 ± .06 | 2.44 ± 0.38 | 0.16 ± .02 | 2.24 ± 0.24 |
| NC (n = 45) | 7.18 ± 0.95 | 0.5 ± 0.1 | 2.52 ± 0.41 | 0.17 ± .03 | 2.33 ± 0.25 |
| Young (n = 10) | 9.16 ± 0.83 | 0.6 ± .08 | 3.03 ± 0.18 | 0.2 ± .03 | 2.79 ± 0.27 |
| *p-value | 0.00052 | 0.00398 | 0.00062 | 0.00092 | <0.00001 |

**% HV (AD + MCI vs. NC) p = 0.001; % AV (AD + MCI vs. NC) p = 0.004; NAA/ml (AD + MCI vs. NC): p = 1. × 10−5
***NAA/mI*% HV (AD + MCI vs. NC): p = 1.9 × 10−8; NAA/mI*% AV (AD + MCI vs. NC): p = 1.4 × 10−9
NAA/ml metabolite ratio, hippocampal volume, and amygdala volume all significantly different between AD vs. NC(*) and AD + MCI vs. NC (**). Combining qMRI and MRS (qMRI × MRS) increases significance by 2-fold (***).

### Example 5

### Conclusion

Volumetric measurements of the amygdala strongly correlate with MRS ratios, as disclosed herein. Hippocampal volume and NAA/mI ratio also have a high degree of correlation. Quantitative MRI and MRS, when combined, provide improved biomarkers over either alone, and can aid in clinical MCI/AD diagnosis.

### Example 6

### Experiments II

### Adding MRS to Drug Monitoring can Reduce Group Size for Clinical Trials

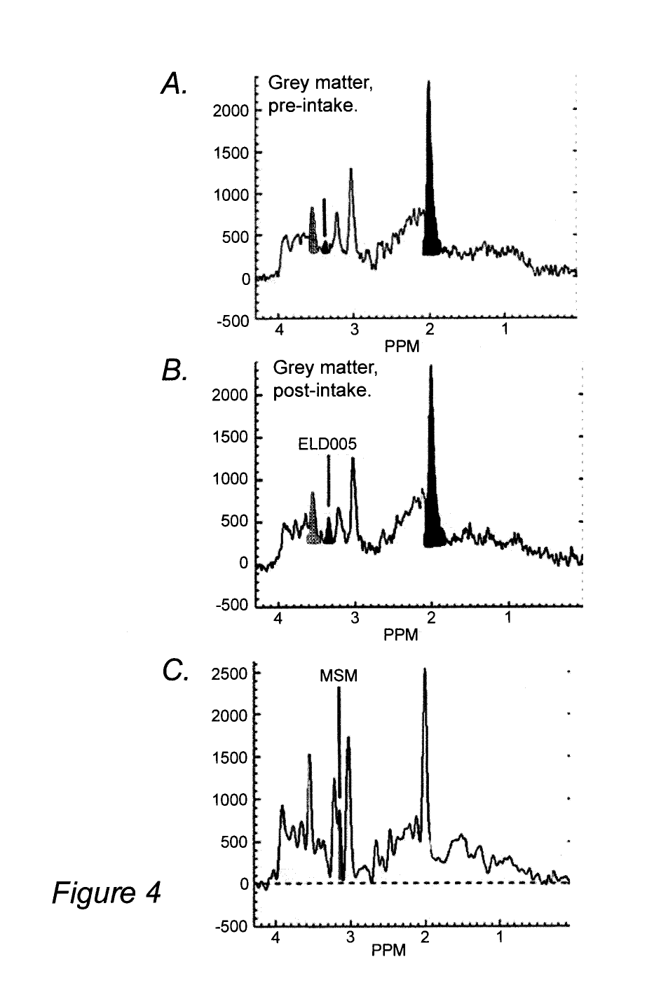

To demonstrate that ¹H MRS can detect AD medications which cross the blood-brain barrier (BBB), spectra from the posterior cingulate gyrus grey matter (GM) are shown for pre-intake and post-intake of ELD005 (FIGS. 4 A and B). There is an obvious increase in the peak at 3.35 ppm. Similarly, ingested methylsulfonylmethane (MSM) can also be observed at 3.15 ppm (FIG. 4C).

### Example 7

### Standard Operating Procedure (SOP) for MRS

A literature survey was completed to establish the longitudinal progression, specificity and sensitivity of MRS for diagnosis of AD and MCI. Experiments were completed on GE 1.5 (N=13), 3.0 Tesla (N=7) and Siemens 3T (University of Kentucky; N=10) clinical MRI scanners to test-retest errors for an automated SOP in MRS acquisitions. Cohort size was established in a potential clinical trial of a drug slowing the progression of AD or MCI, plotting standardized difference (D/sd) vs Power graphically (see Douglas G. Altman. Practical Statistics for Medical Research; Chapman & Hall, London 1990, p. 456, which is incorporated by reference herein in its entirety as though fully set forth). Subjects were examined using automated sequences. PROBE-P TE 35 ms. On the GE systems, 4 spectra were acquired as test, retest I, retest II, shim and newly prescribe voxel. On the Siemens, 1 spectrum was acquired, then the subject was taken out and placed back in the scanner where a new voxel was prescribed.

### Example 8

### Results

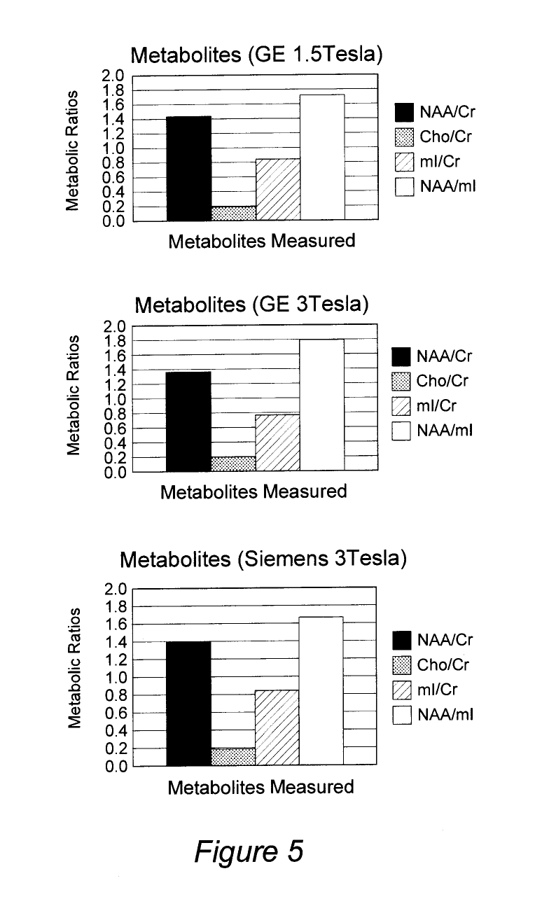

A. SOP: Means on three different scanners are demonstrated in FIG. 5.

### B. Test—Re-Test:

TABLE II

| | | | | T-TESTS | GE 3T |
|---|---|---|---|---|---|
| | % VARIANCE BETWEEN SCANS | | | GE 1.5T | vs Sie- |
| | GE 1.5T | GE 3T | Siemens 3T | vs. 3T | mens 3T |
| NAA/Cr | 3.45 | 3.27 | 3.09 | 0.38 | 0.44 |
| Cho/Cr | 6.51 | 3.75 | 4.82 | 0.03 | 0.26 |
| mI/Cr | 6.15 | 6.8 | 6.28 | 0.35 | 0.42 |
| NAA/mI | 6.40 | 6.91 | 7.70 | 0.34 | 0.39 |
| Group | 100 | 100 | 100 | | |
| Size | | | | | |

Variances of scans on each scanner indicate that the three scanners offer similar precision when the same MRS technique and parameters are applied. This predicts N=100 for group size in MRS trials of AD.

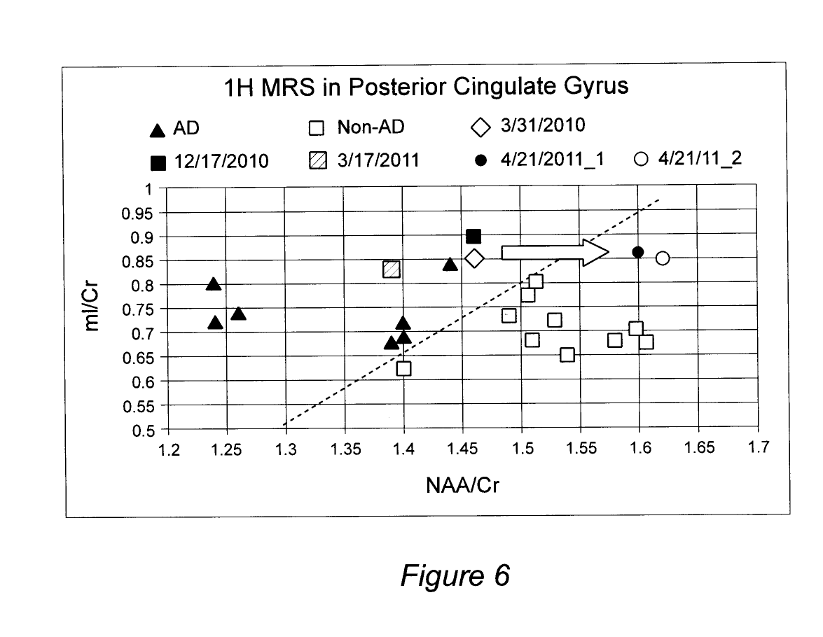

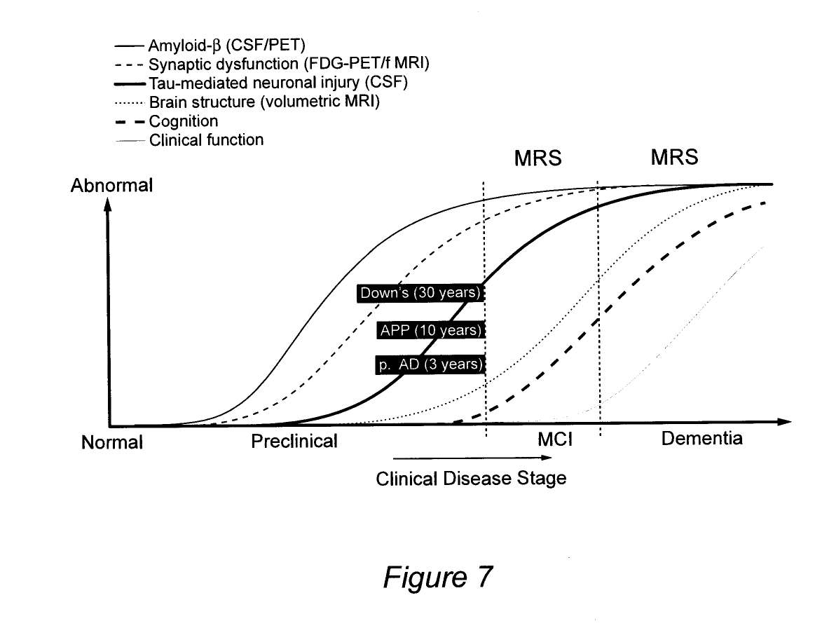

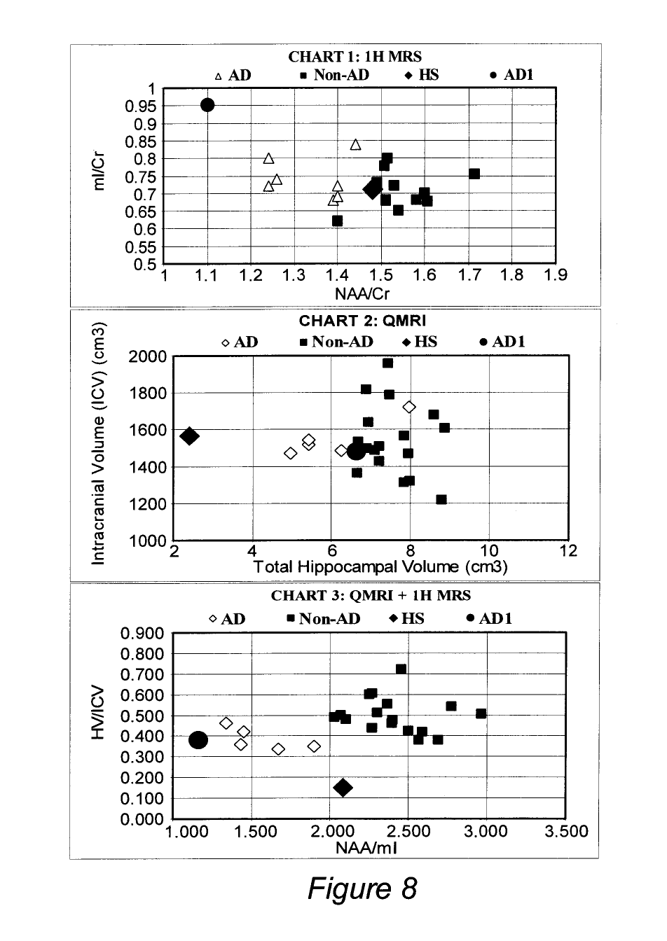

C. Treatment monitoring and longitudinal MRS (FIG. 6).

One patient with clinically diagnosed AD was examined over 13 months using ¹H MRS. The patient was prescribed AB34 and had been taking it for 1 month when she was examined again in April 2011. In this case, although MRS did not detect the actual medication, it detected the direct effect of medications—which is a 14% increase in NAA/Cr in grey matter (arrow) and an 18% increase in NAA/Cr in white matter. This indicates that a group size for a drug which affects NAA/Cr N=10-20.

### Example 9

### Discussion

Group size for monitoring of clinical drug size using SOP of MRS metabolic ratio NAA/mI is very comparable to that claimed for brain MRI. The most commonly stated reason for omission of MRS from drug trials is the absence of a SOP for MRS, and indeed the published literature offers little guidance. Sample size for clinical assays of drugs falls from the N=600 predicted by Schott et al. (see Schott et al. Brain 2010; doi: 10.1093/brain/awq208, which is incorporated herein by reference in its entirety as though fully set forth) to N=100 when NAA/mI error was improved by definition of an SOP, and to ˜20 when NAA/Cr is the biomarker.

### Example 10

### Conclusion

MRS is reliable and reproducible (±6% variance) when rigorous SOP is applied. MRS is an early biomarker which adds specificity in drug trials. MRI and MRS biomarkers appear to be unrelated and thereby amplify the ADNI signal, to further reduce sample sizes for future clinical drug trials in AD.

### Example 11

### Quantitative MRI and Quantitative MRS for Diagnosis of Hippocampal Sclerosis

Hippocampal sclerosis (HS) is commonly misdiagnosed as Alzheimer's disease because of some of the common symptoms associated with each. However, the inventor discovered that while HV is below normal in an individual with HS, the NAA/mI ratio is not. This has significant implications with respect to differentiating between AD and HS, as individuals with AD may or may not have below normal HV measurements, but a below normal NAA/mI ratio is associated with that disease.

Thus, quantitative MRI and quantitative MRS can be performed on an individual suspected of AD or HS in order to determine HV and an NAA/mI ratio, as described above. If the individual has below normal HV, but normal NAA/mI ratio, the individual is diagnosed with HS. If, on the other hand, the individual has a below normal HV, and a below normal NAA/mI ratio, the individual is diagnosed with AD.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventor for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method for diagnosing an individual with Alzheimer's disease or mild cognitive impairment, comprising:
determining a hippocampal volume (HV) and/or an amygdala volume (AV) in the brain of the individual;
determining the ratio of N-acetylasparate (NAA) to myo-inositol (mI) in the brain of the individual; and
diagnosing the individual with Alzheimer's disease or mild cognitive impairment if the HV and/or AV and/or NAA to mI ratio is below that of a healthy individual without Alzheimer's disease or mild cognitive impairment, or diagnosing the individual as not having Alzheimer's disease or mild cognitive impairment if the HV and/or AV and/or NAA to mI ratio is not below that of a healthy individual without Alzheimer's disease or mild cognitive impairment.

2. The method of claim 1, wherein the HV and AV are determined using quantitative magnetic resonance imaging.

3. The method of claim 1, wherein the NAA to mI ratio is determined using quantitative magnetic resonance spectroscopy.

4. A method for diagnosing the progression of Alzheimer's disease or mild cognitive impairment in an individual, comprising:
determining a hippocampal volume (HV) and/or an amygdala volume (AV) in the brain of the individual;
determining the ratio of N-acetylasparate (NAA) to myo-inositol (mI) in the brain of the individual; and
diagnosing the individual as having progressing Alzheimer's disease if the HV and/or AV and/or NAA to mI ratio are lower than previously determined in the same individual, and diagnosing the individual as not having progressing Alzheimer's disease if the HV and/or AV and/or NAA to mI ratio are not lower than previously determined in the same individual.

5. The method of claim 4, wherein the HV and AV are determined using quantitative magnetic resonance imaging.

6. The method of claim 4, wherein the NAA to mI ratio is determined using quantitative magnetic resonance spectroscopy.

7. A method for determining the efficacy of a drug for treating or preventing Alzheimer's disease (AD) or mild cognitive impairment (MCI) in an individual, comprising:
determining a hippocampal volume (HV) and/or amygdala volume (AV) in the brain of the individual;
determining the ratio of N-acetylasparate (NAA) to myo-inositol (mI) in the brain of the individual;
providing a dose of a drug for treating or preventing Alzheimer's disease (AD) or mild cognitive impairment (MCI);
administering the dose of the drug for treating or preventing AD or MCI to the individual;
determining the hippocampal volume (HV) and/or amygdala volume (AV) after administering the drug for treating or preventing AD or MCI to the individual;
determining the ratio of NAA to mI after administering the drug for treating or preventing AD or MCI to the individual;
comparing the HV and/or AV and/or NAA to mI ratio determined prior to administering the drug with the HV and/or AV and/or NAA to mI ratio determined after administering the drug; and
determining the drug has efficacy for treating or preventing AD or MCI if the HV and/or AV and/or NAA to mI ratio has not decreased significantly after administering the drug, and determining the drug does not have efficacy for treating or preventing AD or MCI if the HV and/or AV and/or NAA to mI ratio has decreased significantly after administering the drug.

8. The method of claim 7, wherein the drug is selected from the group consisting of a cholinesterase inhibitor and an N-methyl-D-aspartate (NMDA) receptor antagonist.

9. The method of claim 7, wherein the drug is selected from the group consisting of donepezil, rivastigmine, galantamine, tacrine, and memantine.

10. The method of claim 7, wherein the HV and AV are determined using quantitative magnetic resonance imaging.

11. The method of claim 7, wherein the NAA to nil ratio is determined using quantitative magnetic resonance spectroscopy.

12. A method for determining whether an individual should be included in or excluded from a clinical trial based upon diagnosis or exclusion of Alzheimer's disease or mild cognitive impairment at an early stage, comprising:
determining a hippocampal volume (HV) and/or an amygdala volume (AV) in the brain of the individual;
determining the ratio of N-acetylasparate (NAA) to myo-inositol (mI) in the brain of the individual;
diagnosing the individual with Alzheimer's disease or mild cognitive impairment if the HV and/or AV and/or NAA to mI ratio is below that of a healthy individual without Alzheimer's disease or mild cognitive impairment at an early stage, or diagnosing the individual as not having Alzheimer's disease or mild cognitive impairment if the HV and/or AV and/or NAA to mI ratio is not below that of a healthy individual without Alzheimer's disease or mild cognitive impairment at an early stage; and
determining the individual should be included in the clinical trial if the individual is diagnosed with Alzheimer's disease or mild cognitive impairment at an early stage, and determining the individual should be excluded from the clinical trial if the individual is not diagnosed with Alzheimer's disease or mild cognitive impairment at an early stage.

13. The method of claim 12, wherein the HV and AV are determined using quantitative magnetic resonance imaging.

14. The method of claim 12, wherein the NAA to mI ratio is determined using quantitative magnetic resonance spectroscopy.

15. A method for diagnosing an individual with hippocampal sclerosis (HS), comprising:
determining a hippocampal volume (HV) in the brain of the individual;
determining the ratio of N-acetylasparate (NAA) to myo-inositol (mI) in the brain of the individual; and
diagnosing the individual with HS if the HV is below that of a healthy individual without HS, and the NAA to mI ratio is not below that of a healthy individual without HS.

16. The method of claim 15, wherein the HV is determined using quantitative magnetic resonance imaging.

17. The method of claim 15, wherein the NAA to mI ratio is determined using quantitative magnetic resonance spectroscopy.
